(19) United States
(12) Patent Application Publication
(10) Pub. No.: US 2010/0168844 A1
(43) Pub. Date: Jul. 1, 2010

(54) METHODS AND SYSTEMS FOR REDUCING PARAVALVULAR LEAKAGE IN HEART VALVES

(75) Inventors: Chris Toomes, Orange, CA (US); Marc Nitz, Costa Mesa, CA (US); Tracey Lee, Fremont, CA (US); Bjarne Bergheim, Mission Viejo, CA (US); Rodolfo Quijano, Laguna Hills, CA (US); David Elizondo, Champlin, MN (US)

Correspondence Address: OPPENHEIMER WOLFF & DONNELLY LLP, 45 SOUTH SEVENTH STREET, SUITE 3300, MINNEAPOLIS, MN, 55402, US

(73) Assignee: 3F THERAPEUTICS, INC., LAKE FOREST, CA (US)

(21) Appl. No.: 12/524,531

(22) PCT Filed: Jan. 25, 2008
(86) PCT No.: PCT/US08/52088

§ 371 Date: Jul. 24, 2009

Related U.S. Application Data
(60) Provisional application No. 60/897,669, filed on Jan. 26, 2007.

(51) Int. Cl. A61F 2/24

(52) U.S. Cl. 623/2.18

(57) ABSTRACT

A replacement valve comprises a valve body having an inflow end, an outflow end, and a valve support structure, and a valve cuff surrounding the inflow end of the valve body. The valve support structure surrounds the valve body, and the valve cuff is coupled to the valve support structure. The valve cuff includes a skirt portion and at least one flange coupled to and protruding from the skirt portion, the at least one flange forming a seal around the inflow end of the valve body.

### FIELD OF THE INVENTION

The present invention relates generally to methods and systems for cardiovascular surgery. More particularly, the invention relates to reducing leakage around heart valves.

### BACKGROUND OF THE INVENTION

The transport of vital fluids in the human body is largely regulated by valves. Physiological valves are designed to prevent the backflow of bodily fluids, such as blood, lymph, urine, bile, etc., thereby keeping the body's fluid dynamics unidirectional for proper homeostasis. For example, venous valves maintain the upward flow of blood, particularly from the lower extremities, back toward the heart, while lymphatic valves prevent the backflow of lymph within the lymph vessels, particularly those of the limbs.

Because of their common function, valves share certain anatomical features despite variations in relative size. The cardiac valves are among the largest valves in the body with diameters that may exceed 30 mm, while valves of the smaller veins may have diameters no larger than a fraction of a millimeter. Regardless of their size, however, many physiological valves are situated in specialized anatomical structures known as sinuses. Valve sinuses can be described as dilations or bulges in the vessel wall that houses the valve. The geometry of the sinus has a function in the operation and fluid dynamics of the valve. One function is to guide fluid flow so as to create eddy currents that prevent the valve leaflets from adhering to the wall of the vessel at the peak of flow velocity, such as during systole. Another function of the sinus geometry is to generate currents that facilitate the precise closing of the leaflets at the beginning of backflow pressure. The sinus geometry is also important in reducing the stress exerted by differential fluid flow pressure on the valve leaflets or cusps as they open and close.

Thus, for example, the eddy currents occurring within the sinuses of Valsalva in the natural aortic root have been shown to be important in creating smooth, gradual and gentle closure of the aortic valve at the end of systole. Blood is permitted to travel along the curved contour of the sinus and onto the valve leaflets to effect their closure, thereby reducing the pressure that would otherwise be exerted by direct fluid flow onto the valve leaflets. The sinuses of Valsalva also contain the coronary ostia, which are outflow openings of the arteries that feed the heart muscle. When valve sinuses contain such outflow openings, they serve the additional purpose of providing blood flow to such vessels throughout the cardiac cycle.

When valves exhibit abnormal anatomy and function as a result of valve disease or injury, the unidirectional flow of the physiological fluid they are designed to regulate is disrupted, resulting in increased hydrostatic pressure. For example, venous valvular dysfunction leads to blood flowing back and pooling in the lower legs, resulting in pain, swelling and edema, changes in skin color, and skin ulcerations that can be extremely difficult to treat. Lymphatic valve insufficiency can result in lymphedema with tissue fibrosis and gross distention of the affected body part. Cardiac valvular disease may lead to pulmonary hypertension and edema, atrial fibrillation, and right heart failure in the case of mitral and tricuspid valve stenosis; or pulmonary congestion, left ventricular contractile impairment and congestive heart failure in the case of mitral regurgitation and aortic stenosis. Regardless of their etiology, all valvular diseases result in either stenosis, in which the valve does not open properly, impeding fluid flow across it and causing a rise in fluid pressure, or insufficiency/regurgitation, in which the valve does not close properly and the fluid leaks back across the valve, creating backflow. Some valves are afflicted with both stenosis and insufficiency, in which case the valve neither opens fully nor closes completely.

Because of the potential severity of the clinical consequences of valve disease, numerous surgical techniques may be used to repair a diseased or damaged heart valve. For example, these surgical techniques may include annuloplasty (contracting the valve annulus), quadrangular resection (narrowing the valve leaflets), commissurotomy (cutting the valve commissures to separate the valve leaflets), or decalcification of valve and annulus tissue. Alternatively, the diseased heart valve may be replaced by a prosthetic valve. Where replacement of a heart valve is indicated, the dysfunctional valve is typically removed and replaced with either a mechanical or tissue valve.

In the past, one common procedure has been an open-heart type procedure. However, open-heart valve repair or replacement surgery is a long and tedious procedure and involves a gross thoracotomy, usually in the form of a median sternotomy. In this procedure, a saw or other cutting instrument is used to cut the sternum longitudinally and the two opposing halves of the anterior or ventral portion of the rib cage are spread apart. A large opening into the thoracic cavity is thus created, through which the surgeon may directly visualize and operate upon the heart and other thoracic contents. The patient must typically be placed on cardiopulmonary bypass for the duration of the surgery.

Minimally invasive valve replacement procedures have emerged as an alternative to open-chest surgery. Wikipedia Encyclopedia defines a minimally invasive medical procedure as one that is carried out by entering the body through the skin or through a body cavity or anatomical opening, but with the smallest damage possible to these structures. Two types of minimally invasive valve procedures that have emerged are percutaneous valve procedures and trans-apical valve procedures. Percutaneous valve procedures pertain to making small incisions in the skin to allow direct access to peripheral vessels or body channels to insert catheters. Trans-apical valve procedures pertain to making a small incision in or near the apex of a heart to allow valve access. The distinction between percutaneous valve procedures and minimally invasive procedures is also highlighted in a recent position statement of the Society of Thoracic Surgeons (STS), the American Association for Thoracic Surgery (AATS), and the Society for Cardiovascular Angiography and Interventions (SCAI; Vassiliades Jr. T A, Block P C, Cohn L H, Adams D H, Borer J S, Feldman T, Holmes D R, Laskey W K, Lytle B W, Mack M F, Williams D O. The clinical development of percutaneous heart valve technology: a position statement of the Society of Thoracic Surgeons (STS), the American Association for Thoracic Surgery (AATS), and the Society for Cardiovascular Angiography and Interventions (SCAI). J Thorac Cardiovasc Surg 2005; 129:970-6).

As valves are implanted less and less invasive, the opportunity for suturing the valves around the annulus is reduced. However, a smaller number of sutures may increase the chance of paravalvular leakage (PVL), i.e. leakage around the valve. A smaller number of sutures may also increase the opportunities for migration and valve stability when placed in-vivo.

Tehrani discloses a superior and inferior o-ring for valve implantation in US Patent Application Publication No. 2006/0271172. Such o-rings cover the entire length of the valve and can therefore not easily be placed within the aortic sinus region. The o-rings presented by Tehrani would also block coronary outflow and adversely affect valve dynamics. The non-circular nature of the o-rings also reduces the radial force needed to adequately conform to irregularities within the implantation site, and is thus not optimal for preventing PVL and migration. The large size of the o-rings disclosed by Tehrani is also not practical as they cannot easily be collapsed down, something that is necessary for minimally invasive valve implantation.

While new less invasive valves produce beneficial results for many patients, these valves may not work as well for other patients who have calcified or irregular annuluses because a tight seal may not be formed between the replacement valve and the implantation site. Therefore, what is needed are methods, systems, and devices for reducing paravalvular leakage around heart valves while preventing valve migration and allowing valve collapsibility.

### BRIEF SUMMARY OF THE INVENTION

The present invention provides methods and systems for reducing paravalvular leakage around heart valves. As replacement valve procedures become less and less invasive, the opportunity for suturing the valves around the annulus is reduced. However, minimizing the number of sutures used to secure the replacement valve may increase the chance of paravalvular leakage (PVL), as well as the opportunities for valve migration and valve stability when placed in-vivo.

Leakage associated with a heart valve can be either paravalvular (around the valve) or perivalvular (through the valve). Examples of various heart valves include aortic valves, mitral valves, pulmonary valves, and tricuspid valves. Perivalvular leakage may be reduced by heart valve design. Paravalvular leakage, on the other hand, may be reduced by creating a seal between the replacement heart valve and the implant site to prevent blood from flowing around the replacement heart valve. It is important that the seal between the replacement heart valve and the implant site does not adversely affect the surrounding tissue. Furthermore, it is important that the seal does not affect the flow dynamics around the replacement heart valve. In the case of the aortic valve, it is also important that the seal does not obstruct coronary flow.

Accordingly, it is one object of the present invention to provide methods and devices for preventing paravalvular leakage around a replacement valve, such as a heart valve, while also preventing migration. It should be noted that while reference is made herein to aortic valves, the current invention is not limited to the aortic valve. While replacement valves are typically implanted in native heart valve positions, the replacement valve systems and sealing devices discussed herein may be used to seal any type of in-vivo valve without departing from the intended scope of the present invention.

In one embodiment of the present invention, a valve cuff attachable to a replacement valve to form a seal between the replacement valve and the implant site comprises a skirt and a flange. The skirt may be structured to cover the outside of the replacement heart valve, preferably along the proximal inflow end of the valve. The flange is coupled to the skirt and may be structured to press and seal against the implantation site. In one embodiment of the present invention, the skirt may be scalloped to align with the scallops of the native aortic valve. In one embodiment, the flange may be placed around the outside of the skirt. As such, the flange forms a seal between the skirt and the aorta. In another embodiment, a skirt may be disposed around the outside of the flange.

In one embodiment of the present invention, the cross-sectional area of the flange has a substantially wedge shape, wherein the proximal end of the flange has a larger diameter than the distal end of the valve cuff. A flange whose proximal end is larger than the distal end may be useful to, for example, match the flaring of the aortic valve sinuses.

In another embodiment of the present invention, the cross-sectional area of the flange is substantially circular. In yet another embodiment of the present invention, the cuff comprises two flanges, including one distal flange and one proximal flange. In yet another embodiment of the present invention, the cuff comprises three or more flanges. Utilizing one or more successive flanges may reduce the opportunity for paravalvular leakage. If one flange is not able to completely seal against an annulus irregularity, leakage through this first flange may spill into the volume formed between this first flange and the second flange. The associated pressure drop, blot clotting, and friction may help reduce the opportunity for further leakage through the second flange.

In embodiments where two or more flanges are used, the flanges may have similar cross-sectional areas. Alternatively, at least one of the flanges my have a cross-sectional area having a different size or shape.

When disposed around a replacement heart valve, the protruding flange(s) may be straight (i.e. contained within a plane). Alternatively, the flange(s) may be scalloped to align with the scalloped anatomy of the native aortic valve.

A flange whose cross-sectional area is substantially circular may be made by rolling a flat piece of material, such as a sheet of cloth. One example of a cloth material includes polyester velour. A substantially circular cross-sectional area may also be achieved by folding the sheet of cloth. In yet another embodiment, a generally circular cross-sectional area may be achieved by rolling cloth around another substantially soft material. Examples of such soft materials may include, but are not limited to, silicone, foam, and polymers. In one embodiment of the present invention, cloth may be substituted for other materials such as silicone, polymers, and foam.

It is another object of the present invention to provide a method of preventing paravalvular leakage. Using the valve cuff designs described herein, paravalvular leakage may be reduced by ensuring the cuff is substantially pushed against the aorta, hence forming a tight seal. In one method of implantation, a non self-expanding replacement valve may be expanded into position with a balloon member, thereby pushing the valve cuff against the aorta. In another method of implantation, a self-expanding replacement valve may be deployed into position with a delivery member, thereby pushing the valve cuff against the aorta to create a seal around the valve. In other words, a self-expandable stent contained within the replacement heart valve provides the radial force necessary to push the valve cuff against the aorta. In another method of implantation, the valve cuff may be pushed against the aorta by unrolling the heart valve into position. Regardless of the type of replacement heart valve and the method used to implant the valve, the flange of the valve cuff may contain memory shaped or deformable material that helps tighten the seal with the aorta.

Although many of the above embodiments are described in reference to the aortic valve in the heart, the current invention may also be utilized for procedures related to other valves including, but not limited to, the mitral valve, tricuspid valve, and the pulmonary valve.

The above aspects and other objects, features and advantages of the present invention will become apparent to those skilled in the art from the following description taken together with the accompanying figures.

### BRIEF DESCRIPTION OF THE FIGURES

FIG. 1A illustrates an exemplary valve in an open position during peak flow.

FIG. 1B illustrates the valve of FIG. 1A in a closed position to prevent backflow of the fluid across the valve.

FIG. 2A is a top view illustrating the anatomy of a typical aortic valve.

FIG. 2B is a cross-sectional view of the aortic valve of FIG. 2A.

FIG. 2C is a perspective view of the aortic valve of FIG. 2A showing the inflow end, outflow end, and commissural posts in phantom lines.

FIG. 3 is a schematic representation of the geometry and relative dimensions of the valve sinus region.

FIG. 4 is a perspective view of a valve replacement system in accordance with the present invention, which includes a replacement valve, a valve support structure, and a valve cuff.

FIG. 5 is a perspective view of the replacement valve of FIG. 4.

FIG. 6 is a side view of the valve support structure of FIG. 4 disposed inside a vessel.

FIG. 7 is a side view of the replacement valve system of FIG. 4.

FIG. 8 is a view of the replacement valve system of FIGS. 4 and 7 positioned within an aorta.

FIG. 9 is a cross-sectional view of a portion of the valve cuff of FIGS. 4 and 7.

FIG. 10 is a side view illustrating a replacement valve system having a first alternative embodiment of a valve cuff in accordance with the present invention.

FIG. 11 is a side view illustrating a replacement valve system having a second alternative embodiment of a valve cuff in accordance with the present invention.

FIG. 12 is a side view illustrating a replacement valve system having a third alternative embodiment of a valve cuff in accordance with the present invention.

### DETAILED DESCRIPTION OF THE INVENTION

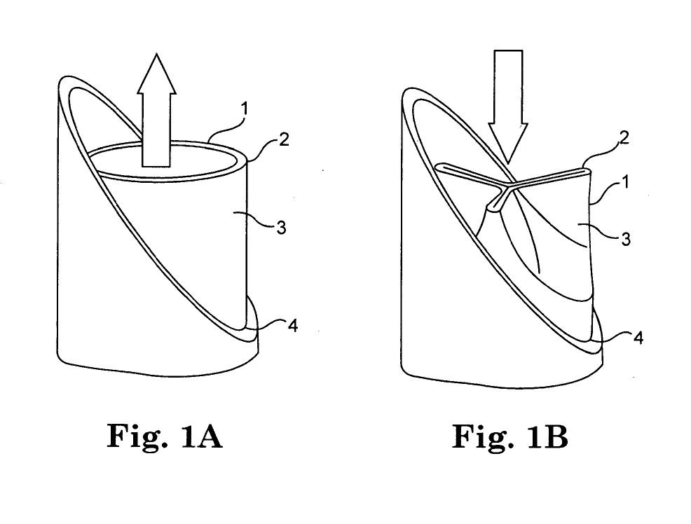

The present invention relates to methods, systems, and devices for reducing paravalvular leakage in heart valves. FIGS. 1A and 1B generally illustrate one exemplary embodiment of a heart valve 1. As illustrated in FIG. 1, valve 1 includes a distal outflow end 2, a plurality of leaflets 3, and a proximal inflow end 4. A typical valve functions similar to a collapsible tube in that it opens widely during systole or in response to muscular contraction to enable unobstructed forward flow across the valvular orifice, as illustrated in FIG. 1A. In contrast, as forward flow decelerates at the end of systole or contraction, the walls of the tube are forced centrally between the sites of attachment to the vessel wall and the valve closes completely as illustrated in FIG. 1B.

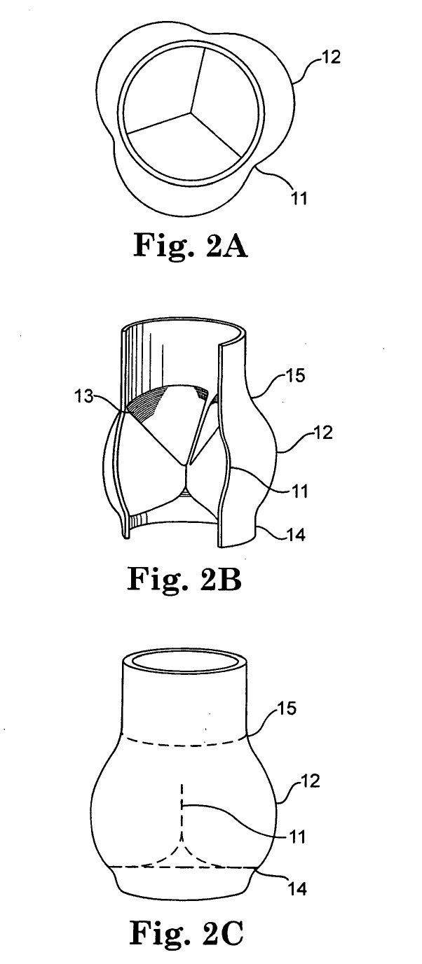

FIGS. 2A, 2B, and 2C illustrate the anatomy of a typical aortic valve. In particular, FIG. 2A shows a top view of a closed valve with three valve sinuses, FIG. 2B shows a perspective sectional view of the closed valve, and FIG. 2C shows a view from outside the vessel wall.

One important consideration in the design of valve replacement systems and devices is the architecture of the valve sinus. Valve sinuses 12 are dilations of the vessel wall that surround the natural valve leaflets. Typically in the aortic valve, each natural valve leaflet has a separate sinus bulge 12 or cavity that allows for maximal opening of the leaflet at peak flow without permitting contact between the leaflet and the vessel wall. As illustrated in FIGS. 2A, 2B, and 2C, the extent of the sinus 12 is generally defined by the commissures 11, vessel wall 13, inflow end 14, and outflow end 15. The proximal intersection between the sinus cavities define the commissures 11.

FIGS. 2B and 2C also show the narrowing diameter of the sinuses at both inflow end 14 and outflow end 15, thus forming the inflow and outflow annuli of the sinus region. Thus, the valve sinuses form a natural compartment to support the operation of the valve by preventing contact between the leaflets and the vessel wall, which, in turn, may lead to adherence of the leaflets and/or result in detrimental wear and tear of the leaflets. The valve sinuses are also designed to share the stress conditions imposed on the valve leaflets during closure when fluid pressure on the closed leaflets is greatest. The valve sinuses further create favorable fluid dynamics through currents that soften an otherwise abrupt closure of the leaflets under conditions of high backflow pressure. Lastly, the sinuses ensure constant flow to any vessels located within the sinus cavities.

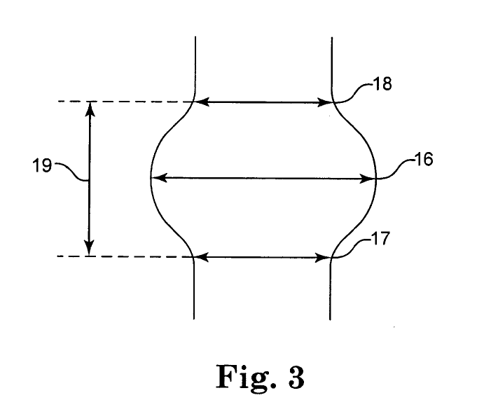

FIG. 3 is a schematic representation of the geometry and relative dimensions of the valve sinus region. As shown in FIG. 3, the valve sinus region is characterized by certain relative dimensions which remain substantially constant regardless of the actual size of the sinuses. Generally, the diameter of the sinus is at its largest at the center of the sinus cavities 16, while there is pronounced narrowing of the sinus region at both the inflow annulus 17 near the inflow end 14 and the outflow annulus 18 near the outflow end 15. Furthermore, the height of the sinus 19 (i.e. the distance between inflow annulus 17 and outflow annulus 18) remains substantially proportional to its overall dimensions. It is thus apparent that the sinus region forms an anatomical compartment with certain constant features that are uniquely adapted to house a valve. The systems and devices of the present invention are designed to utilize these anatomical features of the native sinus region for optimal replacement valve function and positioning.

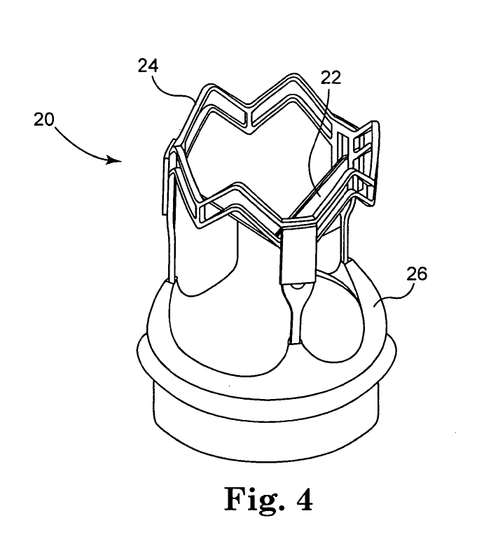

FIG. 4 is a perspective view of a valve replacement system 20 in accordance with the present invention, and includes replacement valve 22, valve support structure 24, and valve cuff 26. As will be discussed in more detail to follow, replacement valve 22 may be attached to valve support structure 24 such that replacement valve 22 resides within the support structure. Valve support structure 24 may be, for example, an expandable and collapsible stent-like structure adapted to be delivered to an implantation site such as a valve sinus. Valve support structure 24 may be either self-expanding or non self-expanding, and may be delivered to the target site via any suitable delivery means as will be appreciated by one skilled in the art. Valve cuff 26 is attachable to the inflow end of replacement valve 22, and is structured to reduce paravalvular leakage around the valve, as well as to reduce migration and increase stability of replacement valve 22 after implantation at the implantation site.

Replacement valve 22 illustrated in FIG. 4 is a tri-leaflet valve. For purposes of example and not limitation, the following discussion will reference only valve 22, it being understood that any stented or stentless replacement valve is contemplated. Similarly, although valve support structure 24 is shown as structured to receive a tri-leaflet valve, those skilled in the art will appreciate that replacement valves having a number of leaflets other than three will correspondingly require a different valve support structure.

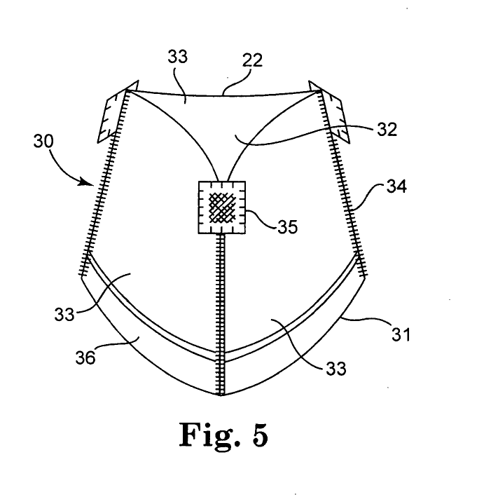

FIG. 5 is a perspective view of replacement valve 22, which represents one exemplary embodiment of a typical, tri-leaflet replacement valve useable with valve replacement system 20 in accordance with the present invention. Replacement valve 22 includes valve body 30 having proximal inflow end 31 and a distal outflow end 32. Valve body 30 includes a plurality of valve tissue leaflets 33 joined by seams 34, wherein each seam 34 is formed by a junction of two leaflets 33. A commissural tab region 35 extends from each seam 34 at the distal end of valve body 30. Inflow end 31 of valve body 30 includes a peripheral edge that may be scalloped or straight. In addition, inflow end 31 of valve body 30 may further comprise reinforcement structure 36 that may be stitched or otherwise attached thereto.

The valve replacement systems and devices of the present invention are not limited, however, to the specific valve illustrated in FIG. 5. For example, although the proximal inflow end 31 of valve body 30 is shown in FIG. 2 with a scalloped peripheral edge, other shapes and configurations are contemplated and within the intended scope of the present invention.

Valve leaflets 33 may be constructed of any suitable material, including but not limited to expanded polytetrafluoroethylene (ePTFE), equine pericardium, bovine pericardium, or native porcine valve leaflets similar to currently available bioprosthetic aortic valves. Other materials may prove suitable as will be appreciated by one skilled in the art.

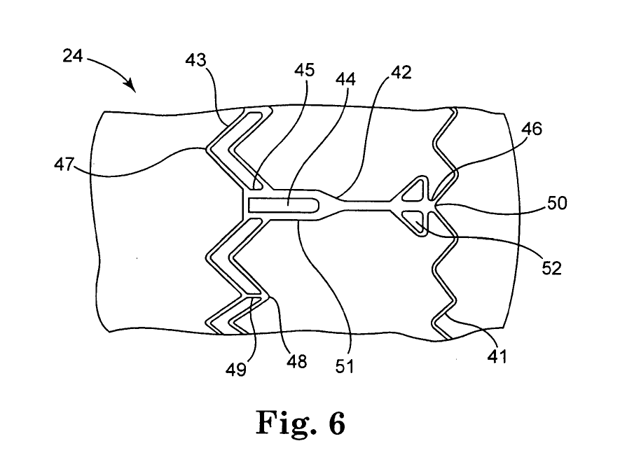

FIG. 6 is a side view of valve support structure 24, which represents one exemplary embodiment of a typical support structure useable with valve replacement system 20 in accordance with the present invention. In general, valve support structure 24 is designed as a collapsible and expandable anchoring structure adapted to support valve 22 distally along commissural tab region 35 and proximally along the proximal inflow end 31. As shown in FIG. 6, valve 22 and valve cuff 26 have been detached from valve support structure 24 so as to focus on the structure and features of the support structure.

Valve support structure 24 has a generally tubular configuration within which replacement valve 22 may be secured, and includes inflow rim 41, support posts 42 and outflow rim 43. Replacement valve 22 may be secured at the proximal inflow end 31 by attachment to inflow rim 41 of support structure 24 and at the distal outflow end 32 via commissural tabs 35 that are threaded through axially extending slots 44, which are formed in support posts 42 that extend longitudinally from inflow rim 41 to outflow rim 43 of valve support structure 24. Thus, distal ends 45 of support posts 42 contact outflow rim 43 of valve support structure 24, whereas proximal ends 46 of support posts 42 contact inflow rim 41 of valve support structure 24.

As shown in FIG. 6, outflow rim 43 of support structure 24 is depicted as comprising a plurality of rings that extend between support posts 42 generally at or above the axially extending slots 44 that reside therein. The plurality of rings of outflow rim 43 are configured in an undulating or zigzag pattern forming peaks 47 and valleys 48, wherein the individual rings remain substantially parallel to one another. The plurality of rings of outflow rim 43 may include a vertical connector element 49 positioned at the center of valleys 48 formed by the undulating or zigzag pattern. Vertical connector element 49 is designed to stabilize support structure 24 and to prevent distortion of the valve during compression and expansion of the support structure. Vertical element 49 extends longitudinally in the axial direction of the cylindrical valve support structure 24.

In the embodiment of valve support structure 24 illustrated in FIG. 6, outflow rim 43 is formed with two rings, while inflow rim 41 is formed with a single ring that extends between support posts 42. However, the number of rings is not important, and numerous other configurations are contemplated.

Both inflow rim 41 and outflow rim 43 of valve support structure 24 are formed with an undulating or zigzag configuration. In various embodiments of valve support structures, inflow rim 41 may have a shorter or longer wavelength (i.e., circumferential dimension from peak to peak) and/or a lesser or greater wave height (i.e., axial dimension from peak to peak) than outflow rim 43. The wavelengths and wave heights of inflow rim 41 and outflow rim 43 may be selected to ensure uniform compression and expansion of valve support structure 24 without substantial distortion. The wavelength of inflow rim 41 is further selected to support the geometry of the inflow end of the valve attached thereto, such as the scalloped inflow end 31 of replacement valve 22 shown in FIG. 5. Notably, as shown in FIG. 6, the undulating or zigzag pattern that forms inflow rim 41 of valve support structure 24 is configured such that proximal ends 46 of vertical support posts 42 are connected to peaks 50 of inflow rim 41. Similarly, the undulating or zigzag pattern that forms outflow rim 43 of support structure 24 is configured such that distal ends 45 of support posts 42 are connected to valleys 48 of outflow rim 43. Locating distal ends 45 of support posts 42 at valleys 48 of outflow rim 43 may prevent the longitudinal extension of outflow rim 43 in the direction of replacement valve 22 secured within the lumen of valve support structure 24 upon compression of the replacement valve assembly 20. As a result, any contact between replacement valve 22 and valve support structure 24 is eliminated. Likewise, locating proximal ends 46 of support posts 42 at peaks 50 of inflow rim 41 may prevent longitudinal extension of inflow rim 41 in the direction of the valve tissue. Thus, compression of replacement valve 22 and valve support structure 24 does not lead to distortion of or injury to the valve.

FIG. 6 further shows that support posts 42 are configured generally in the shape of a paddle with axial slot 44 extending internally within blade 51 of the paddle. Blade 51 of the paddle is oriented toward outflow rim 43 of support structure 24 and connects to outflow rim 43 at a valley 48 of the undulating or zigzag pattern of outflow rim 43. An important function of support posts 42 is the stabilization of valve 22 in general, and in particular the prevention of any longitudinal extension at points of valve attachment to preclude valve stretching or distortion upon compression of replacement valve system 20. Blades 51 of the paddle-shaped support posts 42 may be designed to accommodate commissural tabs 35 of valve 22.

Support posts 42 further comprise triangular shaped elements 52 extending on each side of proximal end 46 of the support post. Triangular shaped elements 52 may be designed to serve as attachments sites for valve cuff 26 and may be designed in different shapes without losing their function. Thus, the particular design of elements 52 shown in FIG. 6 is not critical to the attachment of valve cuff 26, and numerous other designs and shapes are contemplated and within the intended scope of the present invention.

The number of support posts 42 generally ranges from two to four, depending on the number of commissural posts present in the valve sinus. Thus, in a preferred embodiment, valve support structure 24 comprises three support posts for a tri-leaflet replacement valve 22 with a sinus that features three natural commissural posts. Support posts 32 of valve support structure 24 are structured to generally coincide with the natural commissural posts of the valve sinus.

Valve support structure 24 may be formed from any suitable material including, but not limited to, stainless steel or nitinol. The particular material selected for valve support structure 24 may be determined based upon whether the support structure is self-expanding or non self-expanding. For example, preferable materials for self-expanding support structures include shape memory materials, such as nitinol.

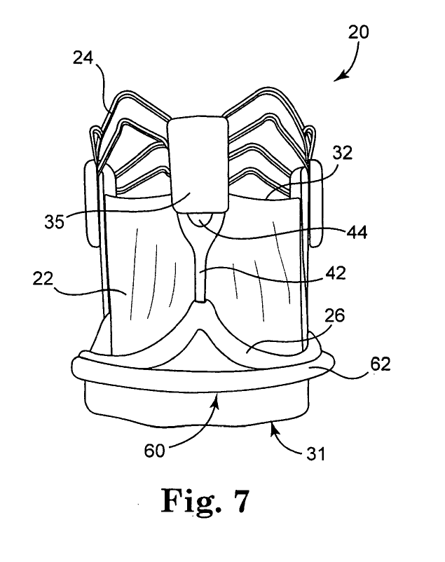

FIG. 7 is a side view illustrating replacement valve system 20 of FIG. 4, which once again includes replacement valve 22, valve support structure 24, and valve cuff 26. As shown in FIG. 7, valve 22 is secured at the proximal inflow end 31 by attachment to inflow rim 41 of valve support structure 24 and at the distal outflow end 32 via commissural tabs 35 that are threaded through axially extending slots 44 formed in support posts 42. Notably, as can be seen in the embodiment shown in FIG. 7, outflow rim 43 of support structure 24 is structured to be longitudinally displaced from the distal outflow end 32 of valve leaflets 33 that reside within the lumen of the tubular valve support structure 24. Thus, contact between valve leaflets 33 and valve support structure 24 is avoided.

The positioning of replacement valve 22 internally to valve support structure 24 with only commissural mounting tabs 35 of replacement valve 22 contacting support posts 42 at the distal outflow end 32 of the valve, while the proximal inflow end 31 of the valve is separated from inflow rim 41 of valve support structure 24 by valve cuff 26, ensures that no part of replacement valve 22 is contacted by valve support structure 24 during operation of valve 22, thereby eliminating wear on valve 22 that may be otherwise result from contact with mechanical elements.

As shown in FIG. 7, valve cuff 26 generally includes skirt 60 and flange 62. As illustrated in FIG. 7, skirt 60 may be structured to cover the outer surface of replacement valve 22, such as along the proximal inflow end 31. In particular, skirt 60 of valve cuff 26 wraps around the entire circumference of replacement valve 22 and valve support structure 24 near the proximal inflow end 31 and inflow rim 41, respectively. Furthermore, as shown in FIG. 7, skirt 60 may have a generally scalloped configuration so as to substantially align with the scallops found in the valve sinus cavity and with the scalloped configuration of replacement valve 22. However, one skilled in the art will appreciate that valve cuffs with non-scalloped skirts are also contemplated and within the intended scope of the present invention.

Skirt 60 of valve cuff 26 is designed to provide numerous benefits when used in conjunction with a replacement valve such as replacement valve 22. First, skirt 60 functions to protect the proximal inflow end 31 of replacement valve 22 from irregularities of a valve annulus such that, for example, calcification remnants or valve remnants left behind after a native valve removal procedure do not come into contact with any portion of replacement valve 22. If otherwise allowed to contact any portion of replacement valve 22, these remnants impose a significant risk of damage to the valve. Second, when positioned adjacent a native valve annulus, skirt 60 provides another source of valve sealing, and also assists valve cuff 26 to conform to irregularities of the valve annulus. Third, once valve cuff 26 is positioned adjacent a valve annulus, skirt 60 allows tissue ingrowth into the valve cuff. Such tissue ingrowth not only improves the seal provided by valve cuff 26, but also helps to anchor the valve cuff to the valve annulus and minimize migration of replacement valve 22 after implantation. Skirt 60 of valve cuff 26 may provide addition benefits other than those previously discussed as will be appreciated by those skilled in the art.

As illustrated in FIG. 7, flange 62 of valve cuff 26 is coupled to skirt 60 and is structured to protrude from replacement valve 22 around the entire circumference of the valve. Once replacement valve system 20 is delivered to an implantation site and deployed, valve support structure 24 exerts a radial force within valve cuff 26 which pushes flange 62 against the aorta, thereby creating a seal to prevent paravalvular leakage and migration of replacement valve 22 within the aorta. For example, in embodiments where valve support structure 24 is formed from a memory shaped metal, the radial force may result from the support structure “springing” back to expanded form after deployment at the implantation site.

Flange 62 of valve cuff 26 is structured for forming a seal between the proximal inflow end 31 of replacement valve 22 and the inflow annulus of the aorta. As previously discussed, when a native valve is removed from a patient's body, irregularities may exist around the inflow annulus of the native valve site. These irregularities may be the result of, for example, natural calcifications or valve remnants left over from extraction of the native valve. Irregularities around the annulus are problematic because they allow paravalvular leakage, which creates a pressure drop across the inflow annulus. As a result of such pressure drop, the replacement valve cannot function in an optimal manner. In the past when irregularities were present, it was difficult to maintain a tight seal between the inflow annulus and the replacement valve. However, flange 62 of valve cuff 26 is structured to conform to irregularities around the inflow annulus, thus improving the seal between replacement valve 22 and the inflow annulus. As a result, paravalvular leakage and the resulting pressure drop across the inflow annulus may be reduced or eliminated.

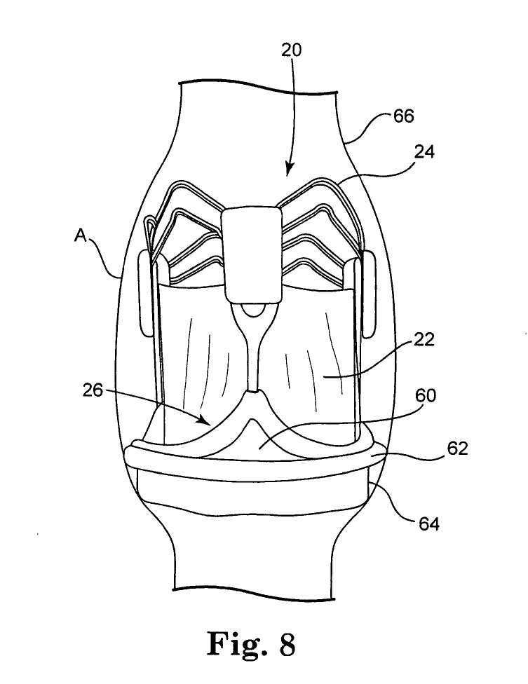

FIG. 8 is a view of replacement valve system 20 positioned within an aorta A, which includes inflow annulus 64 and outflow annulus 66. As shown in FIG. 8, valve support structure 24 has expanded within the sinus cavities of aorta A, thereby forcing flange 62 of valve cuff 26 against inflow annulus 64 of aorta A to form a tight seal between replacement valve 22 and aorta A so as to prevent or at least minimize paravalvular leakage and migration of replacement valve 22 from the implantation site. Thus, with flange 62 in contact with inflow annulus 64, valve cuff 26 acts as a gasket to seal the junction between replacement valve system 20 and aorta A.

In one embodiment, an adhesive may be applied to valve cuff 26 prior to implantation within the aorta. For example, any suitable biocompatible adhesive may be applied to the outer surfaces of skirt 60 and flange 62 to help seal valve cuff 26 to the surrounding tissue of the valve annulus. While not a necessary component of the present invention, biocompatible adhesives may help to provide a tighter seal in order to further reduce paravalvular leakage.

In other embodiments, the flange 62 valve cuff 26 may be constructed with a memory shaped or deformable material disposed within the flange that helps to create a tight seal with the aorta. In particular, the memory shaped or deformable material may be structured to expand once valve cuff 26 is properly positioned at the implantation site. This type of valve cuff flange may be utilized regardless of whether the valve support structure is of the self-expanding or non self-expanding type.

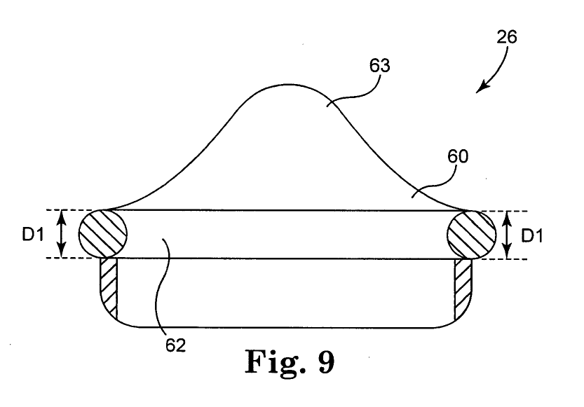

FIG. 9 is a cross-sectional view of a portion of valve cuff 26. As illustrated in FIG. 9, flange 62 of valve cuff 26 has a cross-sectional area having a generally circular shape with a diameter D1. Diameter D1 may preferably be in a range between about 2 mm and about 3 mm, although flanges having other diameters are also contemplated. Furthermore, the diameter D1 of flange 62 remains substantially uniform at all radial positions around flange 62. However, in alternative embodiments, flange 62 may be designed with a generally circular cross-sectional shape having a diameter that does not remain substantially uniform at all radial positions around flange 62, thus forming a flange having an undulating appearance and configuration. Furthermore, flange 62 of valve cuff 26 wraps around the circumference of replacement valve 22 and valve support structure in a substantially flat plane. However, other shapes and configurations of flange 62 are also contemplated, such as a flange that is scalloped.

In one embodiment, both skirt 60 and flange 62 of valve cuff 26 may be formed from a cloth or fabric material. The fabric may comprise any suitable material including, but not limited to, woven polyester such as polyethylene terepthalate, polytetrafluoroethylene (PTFE), or other biocompatible material.

A flange having a cross-sectional area that is substantially circular in shape may be made by numerous methods including, but not limited to, rolling a flat sheet of cloth material to form a cylinder-like member. A substantially circular cross-sectional area may also be achieved for the flange by folding cloth. In yet another embodiment, a generally circular cross-sectional area may be achieved by rolling cloth around another substantially soft material. Such soft materials may include, but are not limited to, silicone, foam, and various polymers. In addition, it is contemplated that these soft materials may be used in a flange embodiment having any other cross-sectional size and shape.

In one exemplary embodiment of assembling valve replacement system 20, skirt 60 and flange 62 are formed as separate components that are coupled together in order to form valve cuff 26. In particular, skirt 60 may initially be positioned around and coupled to valve support structure 24 by any suitable means, such as by suturing. For example, each skirt attachment portion 63 may be wrapped around a corresponding support post 42 of valve support structure 24. Skirt attachment portions 63 may then, for example, be sutured to triangular shaped attachment sites 52 near the proximal ends 46 of each of the support posts 42. Then, flange 62 may be positioned at the desired position around skirt 60 and coupled to the skirt by any suitable means, such as by suturing. Next, replacement valve 22 may be positioned within the inner lumen of valve support structure 24, inserting commissural tab portions 35 of replacement valve 22 through corresponding axially extending slots 44 in support posts 42. Skirt 60 of valve cuff 26, which is positioned circumferentially around inflow rim 41 of valve support structure 24, may then be wrapped around the proximal inflow end 31 of replacement valve 22 and attached to the valve with, for example, sutures. Once attached, skirt 60 and flange 62 are structured to create tight, gasket-like sealing surfaces between replacement valve 22 and the inflow annulus of the aorta. The foregoing represents only one exemplary embodiment of a method of assembling a valve replacement system in accordance with the present invention. Thus, modifications may be made to the number and order of steps as will be appreciate by one skilled in the art.

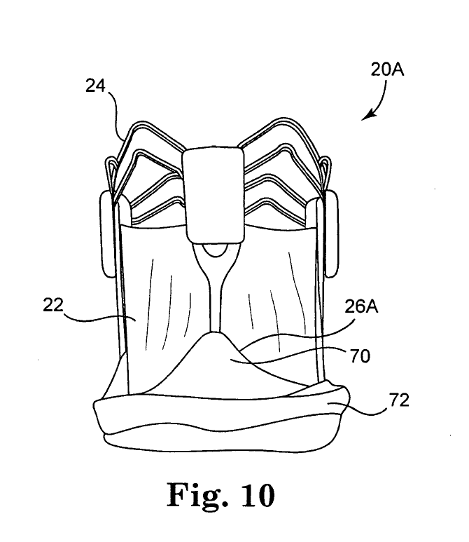

FIG. 10 is a side view illustrating replacement valve system 20A in accordance with the present invention. Replacement valve system 20A generally includes replacement valve 22, support structure 24, and valve cuff 26A, which is a first alternative embodiment of a valve cuff in accordance with the present invention. Valve cuff 26A includes skirt 70 and flange 72. Unlike valve cuff 26 of FIGS. 7-9 which has flange 62 disposed around and coupled to the outer surface of skirt 60, flange 72 of valve cuff 26A is coupled to the inner surface of skirt 70. As such, skirt 72 is designed to form an additional seal between flange 72 and the inflow annulus of the aorta. Thus, one skilled in the art will appreciate that embodiments of valve cuffs having a flange coupled to either the outer surface or the inner surface of a skirt are contemplated and within the intended scope of the present invention.

Skirt 70 is structured to cover the outer surface of replacement valve 22 along the proximal inflow end 31, and has a generally scalloped design so as to substantially align with the scallops found in the valve sinus cavity and with the scalloped configuration of replacement valve 22. Furthermore, flange 72 of valve cuff 26A is structured to surround replacement valve 22 around the entire circumference of the valve. However, unlike the generally circular cross-sectional area of flange 62 of valve cuff 26, flange 72 of valve cuff 26A is designed with a wedge-shaped cross-sectional area. As used herein, “wedge-shape” is intended to mean a flange whose proximal end is smaller that the distal end. Such a configuration may be useful to, for example, match the flaring of the aortic valve sinuses.

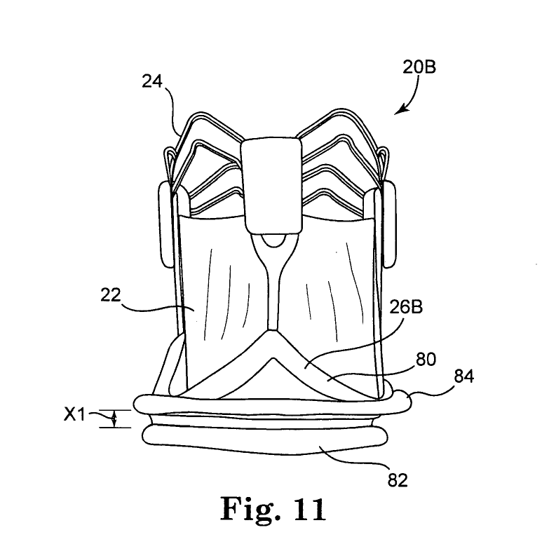

FIG. 11 is a side view illustrating replacement valve system 20B in accordance with the present invention. Replacement valve system 20B generally includes replacement valve 22, support structure 24, and valve cuff 26B, which is a second alternative embodiment of a valve cuff in accordance with the present invention. Valve cuff 26B includes skirt 80, proximal flange 82, and distal flange 84.

Once again, as shown in FIG. 11, skirt 80 is structured to cover the outer surface of replacement valve 22 along the proximal inflow end 31, and has a generally scalloped design so as to substantially align with the scallops found in the valve sinus cavity and with the scalloped configuration of replacement valve 22. Furthermore, both proximal flange 82 and distal flange 84 are structured to protrude from replacement valve 22 around the entire circumference of the valve.

As shown in FIG. 11, proximal flange 82 and distal flange 84 are attached to skirt 80 in close proximity to each other, being spaced apart by a distance X1. Distance X1 may vary depending upon numerous factors such as, for example, the type of valve to which valve cuff 26B is being attached and the particular dimensions of the valve implantation site. For example, in one exemplary embodiment of valve cuff 26B, distance X1 may be about 1 mm.

Furthermore, as shown in FIG. 11, distal flange 84 may be slightly larger than proximal flange 82. In one embodiment, distal flange 84 has a cross-sectional area that is larger than the cross-sectional area of proximal flange 82. Alternatively, both distal flange 84 and proximal flange 82 may have substantially similar cross-sectional areas, but distal flange 84 is coupled to skirt 80 such that it is positioned further away from replacement valve 22 in the radial direction than proximal flange 82. One skilled in the art will appreciate that in embodiments of a valve cuff having two or more flanges coupled to a skirt, the cross-sectional areas of the flanges may have shapes that are the same or different without departing from the intended scope of the present invention. For example, the proximal flange may have a cross-sectional area having a generally circular shape, while the distal flange has a cross-sectional area that is wedge-shaped. Furthermore, although FIG. 11 depicts a distal flange having a cross-sectional area that is larger than the cross-sectional area of a proximal flange, embodiments wherein the cross-sectional area of the proximal flange is larger than the cross-sectional area of the distal flange are also contemplated.

Valve cuffs that utilize two or more successive flanges, such as valve cuff 26B illustrated in FIG. 11, may further reduce the opportunity for paravalvular leakage. If one flange is not able to completely seal against an annulus irregularity, leakage through this first flange may spill into the volume formed between the first flange and the second flange. The associated pressure drop, blot clotting, and friction may help reduce the opportunity for further leakage through the second flange. One skilled in the art will appreciate that additional flanges may be added to a valve cuff as permitted based upon, for example, the size of the native valve site.

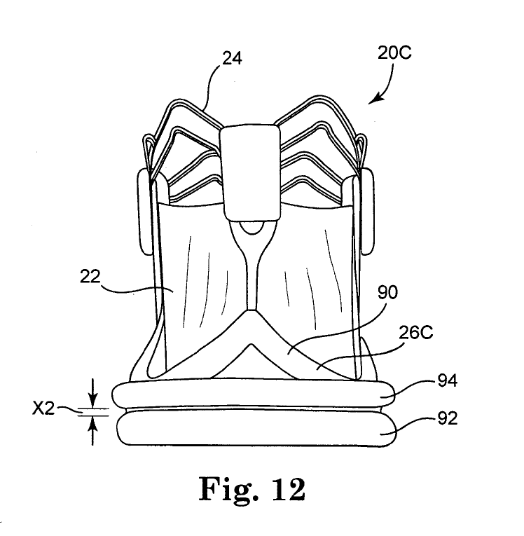

FIG. 12 is a side view illustrating replacement valve system 20C in accordance with the present invention. Replacement valve system 20C generally includes replacement valve 22, support structure 24, and valve cuff 26C, which is a third alternative embodiment of a valve cuff in accordance with the present invention. Valve cuff 26C includes skirt 90, proximal flange 92, and distal flange 94.

Skirt 90 is structured to cover the outer surface of replacement valve 22 along the proximal inflow end 31, and has a generally scalloped design so as to substantially align with the scallops found in the valve sinus cavity and with the scalloped configuration of replacement valve 22. Similar to the flanges previously described, proximal flange 92 and distal flange 94 are coupled to skirt 90 and structured to wrap extend around the entire circumference of replacement valve 22.

As shown in FIG. 12, proximal flange 92 and distal flange 94 are attached to skirt 90 in close proximity to each other, being spaced apart by a distance X2. Distance X2 is less than distance X1 shown in FIG. 11, meaning that proximal and distal flanges 92 and 94 of valve cuff 26C are positioned closer together than proximal and distal flanges 82 and 84 of valve cuff 26B. Once again, distance X2 may vary depending upon numerous factors such as, for example, the type of valve to which valve cuff 26C is being attached and the particular dimensions of the replacement valve implantation site.

Furthermore, as shown in FIG. 12, proximal flange 92 and distal flange 94 have cross-sectional areas that are substantially the same in both size and shape. In particular, both proximal flange 92 and distal flange 94 have cross-sectional areas that are generally circular. Of course, embodiments wherein proximal and distal flanges 92 and 94 have cross-sectional sizes and shapes other than those illustrated in FIG. 12 are also contemplated.

There are several contemplated methods for implanting the valve replacement systems previously described. In the first method, the patient is placed on cardiopulmonary bypass. A small incision is made on the upper sternum to access the ascending aorta. The aorta is clamped and opened to expose the diseased aortic valve, which is excised. The replacement valve system is then inserted within the aorta under direct vision. The valve cuff coupled to the replacement valve thereafter assists in both fixing the valve to the annulus and preventing or reducing paravalvular leakage by forming a tight seal with the aorta.

A second method involves the transcatheter approach. In this method the replacement valve is collapsed or crimped onto a balloon catheter. Preferably, the valve is delivered preloaded on a balloon catheter. This balloon catheter may be inserted via a peripheral artery approach, typically via the femoral artery. In some embodiments, the deployment catheter may be positioned under, for example, fluoroscopic or echocardiographic guidance into the native valve annulus. The valve and the valve cuff are then deployed by expanding the balloon, which pushes the valve cuff against the aorta to form a tight seal for preventing or reducing paravalvular leakage. Successful deployment may be confirmed with, for example, radiographic or echocardiograhic procedures.

In a third method, a self-expanding valve is collapsed and delivered to the aorta in a collapsed state. Once the valve is properly positioned within the aorta, the valve is deployed, thereby allowing the valve to expand into position with the valve cuff pushing against the valve annulus to form a tight seal with the aorta. In such an embodiment, the self-expanding valve includes a self-expanding valve support structure that is structured to provide the radial force necessary to push the cuff against the aorta.

In a fourth method, a non self-expanding valve is “rolled” up and delivered to the aorta. Once property positioned within the aorta, the valve cuff is pushed against the aorta by “unrolling” the replacement valve.

One skilled in the art will appreciate that although only four replacement valve implantation methods are described herein, numerous other methods are possible and within the intended scope of the present invention. Thus, the four exemplary implantation methods are provided for purposes of example and not limitation.

Although the above disclosure focused on a tri-leaflet replacement valve 22, valve cuffs in accordance with the present invention may be used in conjunction with any type of replacement valve of generally similar structure, including but not limited to the heart valves disclosed in U.S. application Ser. No. 10/680,071, U.S. application Ser. No. 11/471,092, and U.S. application Ser. No. 11/489,663, all incorporated herein in their entirety. Therefore, the inventive valve cuff concepts disclosed herein may be applied to valve cuffs structured to function with many other types of replacement valves having any number of leaflets without departing from the spirit and scope of the present invention.

Furthermore, although the above disclosure focuses on valve support structure 24 having an inflow rim 41, an outflow rim 43, and three support posts 42, this particular valve support structure was described merely for purposes of example and not limitation. Thus, valve cuffs in accordance with the present invention may be used in conjunction with any generally tubular, stent-like valve support structure, as will be appreciated by one skilled in the art.

Although the present invention has been described with reference to preferred embodiments, workers skilled in the art will recognize that changes may be made in form and detail without departing from the spirit and scope of the invention.

What is claimed is:

1. A replacement valve system comprising:
a replacement valve having an inflow end, an outflow end, and a plurality of leaflets;
an expandable and collapsible valve support structure having an inflow rim, an outflow rim, and a plurality of support posts extending between the inflow rim and the outflow rim, the replacement valve disposed within the valve support structure; and
a valve cuff surrounding the inflow end of the replacement valve and the inflow rim of the valve support structure, the valve cuff comprising:
a scalloped skirt portion; and
a flange coupled to and protruding from the skirt portion to form a seal around the inflow end of the replacement valve.

2. The replacement valve system of claim 1, further comprising a second flange coupled to and protruding from the skirt potion of the valve cuff.

3. The replacement valve system of claim 2, wherein the flange and the second flange have a generally circular cross-sectional area.

4. The replacement valve system of claim 1, wherein the flange has a generally wedge-shaped cross-sectional area.

5. The replacement valve system of claim 1, wherein a portion of the flange is formed form a shape memory metal alloy.

6. The replacement valve system of claim 1, wherein the flange is coupled to an outer surface of the skirt portion.

7. The replacement valve system of claim 1, wherein the flange is coupled to an inner surface of the skirt portion.

8. The replacement valve system of claim 1, wherein the flange comprises a cloth material wrapped around a foam core.

9. A replacement valve comprising:
a valve body having a proximal inflow end, a distal outflow end, and a plurality of valve leaflets; and
a valve cuff wrapped around the proximal inflow end of the valve body, the valve cuff comprising:
a scalloped skirt portion;
a first flange; and
a second flange;
wherein the first and second flanges are coupled to the skirt portion and are structured to form a seal around the proximal inflow end of the valve body.

10. A valve cuff for a replacement valve comprising:
a scalloped skirt portion structured to surround an inflow end of a replacement valve; and
a plurality of flanges coupled to and protruding from the skirt portion to form a seal around the inflow end of the replacement valve.

11. The valve cuff of claim 10, wherein the flanges have a generally circular cross-sectional area.

12. The valve cuff of claim 10, wherein the skirt portion and the flanges are formed from a cloth material.

13. A replacement valve comprising:
a valve body having an inflow end, an outflow end, and a valve support structure surrounding the valve body; and
a valve cuff surrounding the inflow end of the valve body and coupled to the valve support structure, the valve cuff comprising:
a scalloped skirt portion; and
at least one flange coupled to and protruding from the skirt portion to form a seal around the inflow end of the valve body.

14. The replacement valve of claim 13, wherein the valve cuff comprises two flanges coupled to the skirt portion.

15. The replacement valve of claim 14, wherein the flanges have a substantially similar cross-sectional area.

16. The replacement valve of claim 13, wherein the valve cuff comprises three flanges coupled to the skirt portion.

17. The replacement valve of claim 13, wherein the at least one flange has a generally circular cross-sectional area.

18. The replacement valve of claim 17, wherein the generally circular cross-sectional area has a diameter in a range between about 2 mm and about 3 mm.

19. The replacement valve of claim 13, wherein the at least one flange has a generally wedge-shaped cross-sectional area.

20. The replacement valve of claim 13, wherein a portion of the at least one flange is formed form a shape memory metal alloy.
